(19) United States
(12) Patent Application Publication
(10) Pub. No.: US 2021/0260657 A1
(43) Pub. Date: Aug. 26, 2021

(54) METHOD OF FORMING POROUS COATING LAYER ON SURFACE OF IMPLANT FOR IMPLANTATION INTO LIVING BODY

(71) Applicants: RNX, Inc., Seoul (KR); Industry Academy Cooperation Foundation of Sejong University, Seoul (KR)

(72) Inventors: Do Hyung LIM, Seoul (KR); Tae Yang KWAK, Gwangyang-si (KR); Jin Ju JANG, Seoul (KR)

(21) Appl. No.: 17/182,550

(22) Filed: Feb. 23, 2021

(30) Foreign Application Priority Data
Feb. 26, 2020 (KR) 10-2020-0023926

(51) Int. Cl. B22F 7/04; A61F 2/30; B22F 3/11

(52) U.S. Cl. CPC B22F 7/04; A61F 2/3094; A61F 2/30767; B22F 3/11; B22F 2007/042; A61F 2002/30971; A61F 2002/3093; A61F 2002/30784; A61F 2002/30968

(57) ABSTRACT

A method of forming a porous coating layer on a surface of an implant for implantation into the living body is provided. The method includes a first step of providing an implant base body, which is made of a material including a metal component, and a second step of sintering metal powder on a surface of the implant base body using rapid prototyping. In the second step, a laser beam irradiation tool necessary for the rapid prototyping repeatedly moves along a predetermined movement path to sinter the metal powder, which is sprayed along the predetermined movement path, on the surface of the implant base body, and an inflection point is present between semicircular curved section paths in the predetermined movement path.

### CROSS-REFERENCE TO RELATED APPLICATION

This application claims priority to and the benefit of Korean Patent Application No. 10-2020-0023926, filed on Feb. 26, 2020, the disclosure of which is incorporated herein by reference in its entirety.

### BACKGROUND

### 1. Field of the Invention

The present disclosure relates to a method of forming a porous coating layer on a surface of an implant for implantation into the living body, and more particularly, to a method capable of manufacturing an orthopedic implant with improved osseointegration.

### 2. Discussion of Related Art

In recent years, with the progression of an aging society, the incidence of arthritis has expanded, and diseases such as degenerative arthritis have spread rapidly due to an increase in the obese population, etc.

Thus, the market size for artificial joints has been increasing, and there has been a growing interest in technologies such as personalized artificial joints and porous surface treatment that are intended to minimize side effects such as complications.

Here, artificial joints may be represented as orthopedic implants, and orthopedic implants generally promote bone growth, that is, osseointegration, on an implant base through coating of a porous structure.

An orthopedic implant on which a coating layer having a porous structure is formed may be manufactured using various methods such as diffusion bonding disclosed in Japanese Unexamined Patent Application Publication No. 2008-194463.

As disclosed in Japanese Unexamined Patent Application Publication No. 2008-194463, a conventional method of manufacturing an orthopedic implant, on which a coating layer having a porous structure is formed, by diffusion bonding includes placing a porous structure on a prefabricated implant base and then applying a predetermined pressure to the porous structure under a predetermined temperature to bond the porous structure and the implant base to each other.

However, according to the conventional method using diffusion bonding, since the porosity around the surface of the coating layer is decreased, the degree of osseointegration is reduced, and simultaneously, the bonding strength between the coating layer and the implant base is not guaranteed. As a result, there is a serious problem that defective products may be mass-produced.

### SUMMARY OF THE INVENTION

The present disclosure is directed to providing a method of forming a porous coating layer on a surface of an implant for implantation into the living body, the method capable of increasing the porosity of the porous coating layer, which is formed on the surface of the implant for implantation into the living body, to promote osseointegration in pores and capable of increasing the adhesion between particles constituting the porous coating layer and the adhesion between an implant base body and the particles to improve corrosion resistance and wear resistance.

According to the present disclosure, a method of forming a porous coating layer on a surface of an implant for implantation into the living body includes a first step of providing an implant base body, which is made of a material including a metal component, and a second step of sintering metal powder on a surface of the implant base body using rapid prototyping, wherein, in the second step, a laser beam irradiation tool necessary for the rapid prototyping repeatedly moves along a predetermined movement path to sinter the metal powder, which is sprayed along the predetermined movement path, on the surface of the implant base body so that the porous coating layer is formed on the surface of the implant base body, the predetermined movement path includes a semicircular first curved section path and a semicircular second curved section path (an inflection point is present between the first curved section path and the second curved section path), and in a process in which the movement path of the laser beam irradiation tool is changed from the first curved section path to the second curved section path, the first curved section path and the second curved section path reduce a decrease in a movement speed of the laser beam irradiation tool to reduce a difference in energy yield per unit area of the surface, which is due to a laser beam irradiated by the laser beam irradiation tool, so that a decrease in a porosity of the porous coating layer formed on the surface of the implant base body is prevented.

In the method of forming the porous coating layer on the surface of the implant for implantation into the living body, the first curved section path and the second curved section path may be symmetrical with respect to the inflection point.

In the method of forming the porous coating layer on the surface of the implant for implantation into the living body, the predetermined movement path may be provided to be a mirror image of a movement path for a subsequent row with respect to a virtual boundary line therebetween.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is an exemplary view of an implant for implantation into the living body that is manufactured using a method of forming a porous coating layer on a surface of the implant for implantation into the living body according to the present disclosure;

FIG. 2 is a conceptual diagram relating to rapid prototyping used in the method of forming the porous coating layer on the surface of the implant for implantation into the living body according to the present disclosure; and

FIGS. 3A to 8 are views for describing a movement path of a laser beam irradiation tool for implementing the method of forming the porous coating layer on the surface of the implant for implantation into the living body according to the present disclosure.

### DETAILED DESCRIPTION OF EXEMPLARY EMBODIMENTS

Hereinafter, exemplary embodiments of the present disclosure will be described in detail with reference to the drawings. However, the idea of the present disclosure is not limited to embodiments proposed herein, and those of ordinary skill in the art who understand the idea of the present disclosure may easily propose another less advanced invention or another embodiment included within the scope of the idea of the present disclosure by adding, changing, or omitting an element within the scope of the same idea. However, these should also be construed as belonging to the scope of the idea of the present disclosure.

In addition, like reference numerals will be used to describe like elements having the same functions within the scope of the same idea illustrated in the drawings of each embodiment.

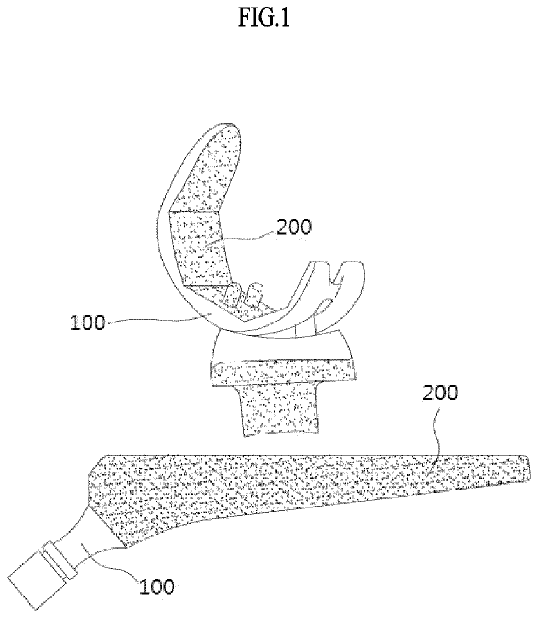

FIG. 1 is an exemplary view of an implant for implantation into the living body that is manufactured using a method of forming a porous coating layer on a surface of the implant for implantation into the living body according to the present disclosure. FIG. 1 is a view illustrating an artificial hip joint and an artificial knee joint.

Referring to FIG. 1, the artificial hip joint and the artificial knee joint are typical orthopedic implants in which a porous coating layer 200 may be formed on a surface of an implant base body 100.

Here, the implant base body 100 may be made of a metal component, for example, a metal mainly consisting of cobalt chromium (CoCr), and may be manufactured using rapid prototyping, which is so-called three-dimensional (3D) printing.

However, a method of manufacturing the implant base body 100 is not limited to the method mentioned above.

The porous coating layer 200 may be made of a metal component, for example, a metal mainly consisting of titanium (Ti), and may include numerous pores.

The numerous pores included in the porous coating layer 200 may be connected to each other, and the pores act as factors that improve osseointegration.

In order to secure the connectivity between the pores included in the porous coating layer 200, to implement the optimum porosity for improvement of osseointegration, to improve the adhesion between Ti particles constituting the porous coating layer 200, and to improve the adhesion between the implant base body 100 and the porous coating layer 200, the porous coating layer 200 may be implemented using rapid prototyping, which is so-called 3D printing, on the implant base body 100 as a base. This will be described in detail below.

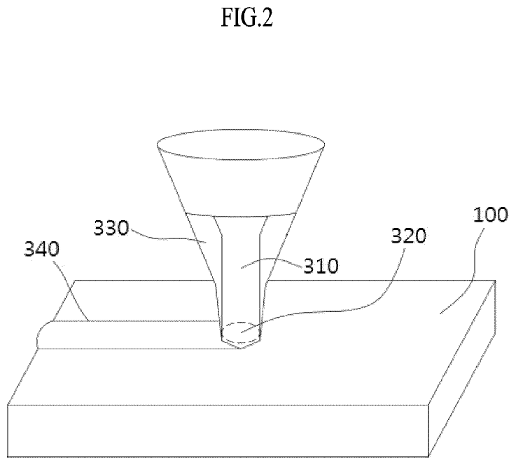

FIG. 2 is a conceptual diagram relating to rapid prototyping used in the method of forming the porous coating layer on the surface of the implant for implantation into the living body according to the present disclosure.

Rapid prototyping is a processing method capable of directly producing 3D products or tools necessary for product production in a short time using geometrical data of a 3D model stored in a computer, such as 3D computer-aided design (CAD) data, computerized tomography (CT) or magnetic resonance imaging (MRI) data, and digital data acquired by a 3D scanner and may be a concept including selective laser sintering (SLS), direct metal laser sintering (DMLS), selective laser melting (SLM), electron beam melting (EBM), laser-aided direct metal tooling (DMT), laser-engineered net shaping (LENS), direct metal deposition (DMD), directed focused deposition (DED), direct metal fab (DMF), and the like.

In more detail, referring to FIG. 2, rapid prototyping is a method of forming the porous coating layer 200 using metal powder such as Ti, on the implant base body 100 made of CoCr or the like. A surface of the implant base body 100 is irradiated with a laser beam 310 along a predetermined path to locally form a melt pool 320, and simultaneously, metal powder 330 is supplied from the outside to form a metal powder layer 340 on the surface of the implant base body 100.

The metal powder layer 340 is formed along the predetermined path. The metal powder layer 340 may also be formed due to moving the implant base body 100 along the predetermined path in a state in which the laser beam 310 is fixed.

Here, the porous coating layer 200 is implemented as the metal powder layer 340 is formed along the predetermined path and then the metal powder layers is repeatedly stacked thereon so as to be formed again, and the pores are implemented by portions to which the metal powder is not supplied.

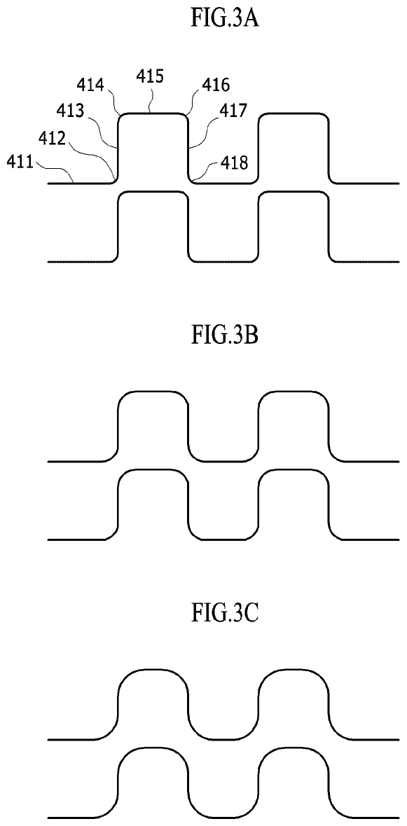

In order to improve osseointegration, the size of pores provided in the porous coating layer 200, the connectivity between the pores, the optimum porosity, the adhesion between the Ti particles, and the like are implemented by an optimized predetermined path. This will be described in detail with reference to FIGS. 3A to 8.

FIGS. 3A to 8 are views for describing a movement path of a laser beam irradiation tool for implementing the method of forming the porous coating layer on the surface of the implant for implantation into the living body according to the present disclosure.

First, the method of forming the porous coating layer on the surface of the implant for implantation into the living body according to the present disclosure may include a first step of providing the implant base body 100, which is made of a material including a metal component, and a second step of sintering metal powder on a surface of the implant base body 100 using rapid prototyping.

Here, as described above, the implant base body 100 may be made of a material such as CoCr, and the metal powder may be Ti powder.

In the second step, a laser beam irradiation tool necessary for the rapid prototyping may repeatedly move along a predetermined movement path to sinter the metal powder, which is sprayed along the predetermined movement path, on the surface of the implant base body 100 so that the porous coating layer 200 is formed on the surface of the implant base body 100.

Of course, in the second step, the implant base body 100 may repeatedly move along the predetermined movement path in a state in which the laser beam irradiation tool is fixed.

As illustrated in FIGS. 3A to 3C, the predetermined movement path may include a first linear section path 411, a first curved section path 412, a second linear section path 413, a second curved section path 414, a third linear section path 415, a third curved section path 416, a fourth linear section path 417, and a fourth curved section path 418.

The first curved section path 412 may cause the second linear section path 413 to be changed by a predetermined first angle from the first linear section path 411.

The second curved section path 414 may cause the third linear section path 415 to be changed by a predetermined second angle from the second linear section path 413.

The third curved section path 416 may cause the fourth linear section path 417 to be changed by a predetermined third angle from the third linear section path 415.

The fourth curved section path 418 may cause the first linear section path 411 to be changed by a predetermined fourth angle from the fourth linear section path 417.

Here, the first angle, the second angle, the third angle, and the fourth angle may be 90° but are not necessarily limited thereto and may be an acute angle or an obtuse angle.

The first curved section path 412, the second curved section path 414, the third curved section path 416, and the fourth curved section path 418 may reduce a decrease in a movement speed of the laser beam irradiation tool in a process in which the movement path of the laser beam irradiation tool is changed by the first angle, the second angle, the third angle, and the fourth angle, respectively.

When the movement path of the laser beam irradiation tool is changed by the first angle, the second angle, the third angle, and the fourth angle without the first curved section path 412, the second curved section path 414, the third curved section path 416, and the fourth curved section path 418, the movement speed of the laser beam irradiation tool is inevitably decreased, and thus, in portions where the movement speed is decreased, the energy yield per unit area of the surface which is due to the laser beam is increased, and the amount of sintered metal powder also increases correspondingly.

As a result, it becomes difficult to implement the optimized pore size and porosity. This is the reason why the present disclosure includes the first curved section path 412, the second curved section path 414, the third curved section path 416, and the fourth curved section path 418, and due to the curved section paths 412, 414, 416, and 418, a decrease in the porosity of the porous coating layer 200 formed on the surface of the implant base body 100 may be prevented to improve osseointegration.

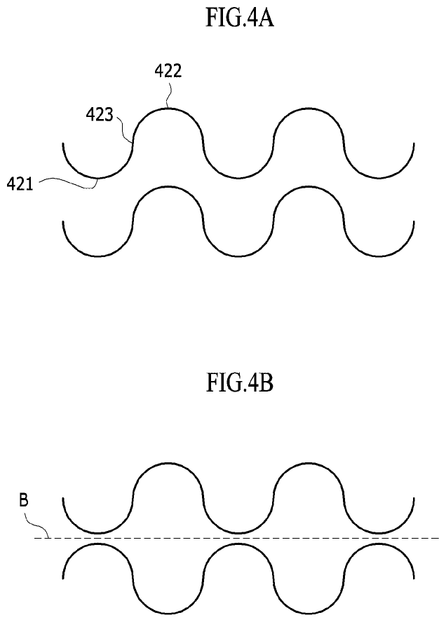

Meanwhile, as illustrated in FIGS. 4A to 4B, the predetermined movement path may include a semicircular first curved section path 421 and a semicircular second curved section path 422, and an inflection point may be present between the first curved section path 421 and the second curved section path 422.

Here, the first curved section path 421 and the second curved section path 422 may reduce a decrease in the movement speed of the laser beam irradiation tool in a process in which the movement path of the laser beam irradiation tool is changed from the first curved section path 421 to the second curved section path 422 to reduce a difference in the energy yield per unit area of the surface, which is due to the laser beam irradiated by the laser beam irradiation tool, so that a decrease in the porosity of the porous coating layer 200 formed on the surface of the implant base body 100 is prevented.

The first curved section path 421 and the second curved section path 422 may be symmetrical with respect to the inflection point 423, and as illustrated in FIG. 4B, the predetermined movement path may be provided to be a mirror image of a movement path for a subsequent row with respect to a virtual boundary line B therebetween.

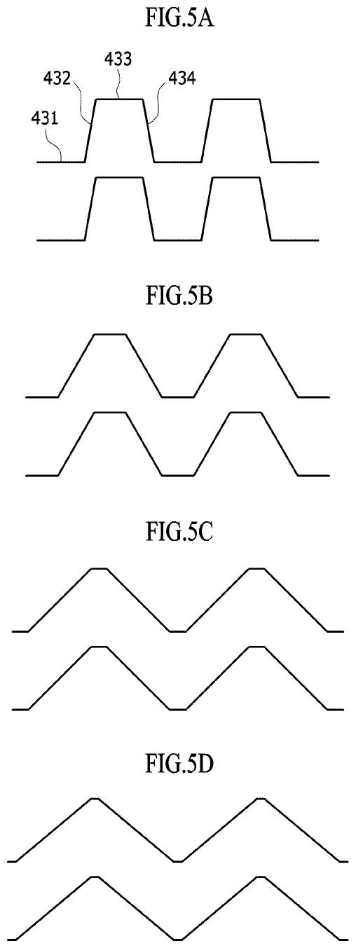

Meanwhile, as illustrated in FIGS. 5A to 5D, the predetermined movement path may include a first linear section path 431, a second linear section path 432, a third linear section path 433, and a fourth linear section path 434.

The second linear section path 432, the third linear section path 433, and the fourth linear section path 434 may be formed within an obtuse angle range with respect to the first linear section path 431, the second linear section path 432, and the third linear section path 433, respectively.

Thus, a decrease in the movement speed of the laser beam irradiation tool in a process in which the movement path of the laser beam irradiation tool is changed may be reduced to reduce a difference in the energy yield per unit area of the surface, which is due to the laser beam irradiated by the laser beam irradiation tool, so that a decrease in the porosity of the porous coating layer formed on the surface of the implant base body is prevented.

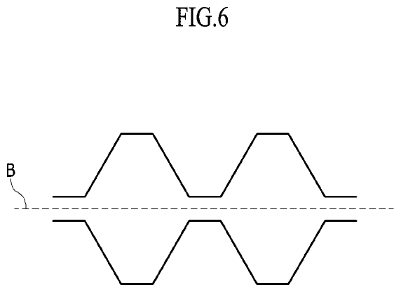

Meanwhile, each of the predetermined movement paths illustrated in FIGS. 3A to 5D may be provided as a mirror image of a movement path for a subsequent row with respect to a virtual boundary line B therebetween. An example thereof is illustrated in FIG. 6.

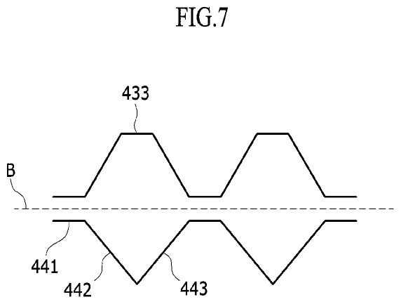

Meanwhile, as illustrated in FIG. 7, a movement path of the laser beam irradiation tool for a subsequent row of the predetermined movement path may be repeatedly formed along a predetermined subsequent movement path, and the predetermined subsequent movement path may include a fifth linear section path 441, a sixth linear section path 442, and a seventh linear section path 443.

The sixth linear section path 442 may be formed within an obtuse angle range with respect to the fifth linear section path 441, and the seventh linear section path 443 may be formed within an acute angle range with respect to the sixth linear section path 442.

An inflection point between the sixth linear section path 442 and the seventh linear section path 443 may be located in a vertical region within the range of the third linear section path 433.

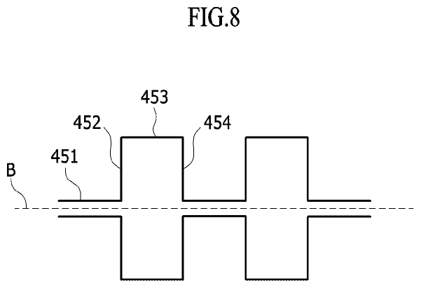

Meanwhile, as illustrated in FIG. 8, the predetermined movement path may include a first linear section path 451, a second linear section path 452, a third linear section path 453, and a fourth linear section path 454, and the second linear section path 452, the third linear section path 453, and the fourth linear section path 454 may be formed within a right angle range with respect to the first linear section path 451, the second linear section path 452, and the third linear section path 453, respectively.

Also, the predetermined movement path may be provided to be a mirror image of a movement path for a subsequent row with respect to a virtual boundary line B therebetween.

Using a method of forming a porous coating layer on a surface of an implant for implantation into the living body according to the present disclosure, the porosity of the porous coating layer, which is formed on the surface of the implant for implantation into the living body, can be increased to promote osseointegration in pores.

Also, the adhesion between particles constituting the porous coating layer and the adhesion between an implant base body and the particles can be increased to improve corrosion resistance and wear resistance.

In addition, since the porous coating layer is formed using rapid prototyping, accuracy can be improved, and there is an advantage in terms of manufacture.

The configurations and features of the present disclosure have been described above using the embodiments according to the present disclosure, but the present disclosure is not limited thereto, and it should be apparent to those of ordinary skill in the art, to which the present disclosure pertains, that various changes or modifications may be made within the idea and scope of the present disclosure. Note that such changes or modifications fall within the scope of the attached claims.

What is claimed is:

1. A method of forming a porous coating layer on a surface of an implant for implantation into a living body, the method comprising:
a first step of providing an implant base body which is made of a material including a metal component; and
a second step of sintering metal powder on a surface of the implant base body using rapid prototyping,
wherein, in the second step, a laser beam irradiation tool necessary for the rapid prototyping repeatedly moves along a predetermined movement path to sinter the metal powder, which is sprayed along the predetermined movement path, on the surface of the implant base body so that the porous coating layer is formed on the surface of the implant base body,
the predetermined movement path includes a semicircular first curved section path and a semicircular second curved section path,
an inflection point is present between the first curved section path and the second curved section path, and
in a process in which the movement path of the laser beam irradiation tool is changed from the first curved section path to the second curved section path, the first curved section path and the second curved section path reduce a decrease in a movement speed of the laser beam irradiation tool to reduce a difference in energy yield per unit area of the surface, which is due to a laser beam irradiated by the laser beam irradiation tool, so that a decrease in a porosity of the porous coating layer formed on the surface of the implant base body is prevented.

2. The method of claim 1, wherein the first curved section path and the second curved section path are symmetrical with respect to the inflection point.

3. The method of claim 2, wherein the predetermined movement path is provided to be a mirror image of a movement path for a subsequent row with respect to a virtual boundary line therebetween.
